(19) United States
(12) Patent Application Publication
(10) Pub. No.: US 2025/0177159 A1
(43) Pub. Date: Jun. 5, 2025

(54) EXPANDABLE INTERVERTEBRAL SPACER

(71) Applicant: The Feinstein Institutes for Medical Research, Manhasset, NY (US)

(72) Inventors: Todd Goldstein, Albertson, NY (US); Sohrab Virk, New Hyde Park, NY (US)

(21) Appl. No.: 18/964,145

(22) Filed: Nov. 29, 2024

Related U.S. Application Data
(60) Provisional application No. 63/605,042, filed on Dec. 1, 2023.

(51) Int. Cl. A61F 2/44

(52) U.S. Cl. CPC A61F 2/442; A61F 2/4455

(57) ABSTRACT

An expandable interbody spacer for intervertebral fusion is provided. The interbody spacer includes an anterior spacer, at least two anterior hinge members having a first end and a second end, a first lateral spacer, a second lateral spacer, at least two posterior hinge members having a first end and a second end, and a posterior spacer. The anterior spacer is operatively coupled to the first ends of the anterior hinge members. The second end of the first anterior hinge member is operatively coupled to the first lateral spacer. The second end of the second anterior hinge member is operatively coupled to the second lateral spacer. The posterior spacer is operatively coupled to the first ends of the posterior hinge members. The second end of the first posterior hinge member is connected to the first lateral spacer. The second end of the second posterior hinge member is connected to the second lateral spacer. The interbody spacer is configured to expand bilaterally in a medial-lateral direction such that the first and second lateral spacers engage the lateral most aspects of the adjacent vertebrae.

The present application claims priority under 35 U.S.C. § 119 to U.S. Provisional Patent Application No. 63/605,042, filed on Dec. 1, 2023, which is incorporated herein by reference.

### BACKGROUND

### Field

The present disclosure generally relates to a device for facilitating intervertebral fusion and to methods of using such a device. More particularly, the present disclosure relates to an expandable interbody device capable of being inserted between adjacent vertebrae to facilitate interbody fusion of the spine. Use of such a device may obviate the need for supplemental pedicle screw fixation.

### Background

The spine is formed by vertebrae separated by discs. The vertebrae provide the support and structure of the spine while the spinal discs act as cushions or “shock absorbers.” Spinal discs also contribute to the flexibility and motion of the spinal column. Over time, the vertebrae and discs may become diseased or infected or may develop deformities which result in loss of structural integrity. This may cause the disc to bulge or to flatten. Impaired vertebrae and discs may result in reduced biomechanical support and are often associated with chronic back pain.

Non-surgical treatments, such as medication, rehabilitation, and exercise can be effective in improving stability and reducing pain. In some cases, however, such treatment may fail to relieve the symptoms. In addition, certain disorders, for example, herniated or bulging discs, may not be amenable to non-surgical treatment. Surgical treatment of these spinal disorders may require fusion, fixation, correction, discectomy, laminectomy and implantable prosthetics. As part of these surgical treatments, spinal constructs, for example, bone fasteners, pedicle screws, and spinal rods, may be connected between adjacent vertebral bodies to fix those vertebrae together. In some surgical procedures, the disc is removed and an interbody device is introduced between adjacent vertebrae in the interbody space to properly space the vertebral bodies and provide stability for augmentation of fusion

More recently, interbody devices have been introduced that provide additional capability beyond static spacing of the vertebral bodies. For example, some devices have expansion capability such that the implant may be introduced to the interbody space in a collapsed state and then expanded to produce additional spacing and, in some cases, introduce or restore curvature to the spine by expanding to different thicknesses along selected portions of the interbody space. One problem with known expandable interbody prostheses is that they have limited expansion directions. More particularly, many existing designs expand in only one direction.

Another problem of current interbody devices is that they may lead to subsidence where the interbody device collapses through the bony endplate and into the vertebral body of the vertebra adjacent to the device. In practice, when subsidence occurs, the device tends to collapse through the end plate of the vertebra above the device. Subsidence typically results in a loss of height restoration, a decrease or loss of lordosis, pain due to endplate fracture and loss of adequate support.

One cause of subsidence is the location of the contact points of the interbody device with the vertebral endplate. Many current interbody devices have contact predominantly or exclusively on the cancellous portion of the vertebral endplate. Such devices may have less contact with the stronger cortical bony rim of the endplate. Contact of the interbody device with the softer portion of the vertebral endplates may increase the risk of subsidence and further complicate postoperative recovery.

An additional problem exists related to subsidence of spinal surfaces due to existing interbody devices having inadequately sized load-bearing surfaces. In the case of expandable devices, the loads on the load-bearing surfaces, including loads generated during expansion of the implant, are often significant. An expandable implant with relatively large contact areas may be needed to bear the loads of a patient's activities of daily living and the loads generated during implant expansion. Increasing the surface area of the device may increase the overall size of the device and may require a larger surgical incision, potentially increasing the patient's exposure to infection.

A further problem with known interbody devices is the migration and movement of these devices in the postoperative period. Known interbody devices may not sit securely between the vertebrae and, as a result, move or migrate. Such migration may increase the risk of subsidence, non-union of the fusion, and neurologic injury.

Movement of adjacent vertebrae relative to one another may increase the risk that the interbody device will migrate. Relative movement between adjacent vertebrae may be limited by securing the vertebrae with one another using pedicle screws and rods. By fixing the vertebrae to one another, motion of the vertebrae is limited, which reduces the tendency for an interbody spacer between the vertebrae to migrate. However, even where pedicle screws fix the two adjacent vertebrae together, the interbody device may still be subject to migration and movement.

Currently the majority of posterior cervical and almost all anterior and posterior lumbosacral and thoracic fusion techniques are supplemented by securing vertebral bodies using pedicle screws. Pedicle screw placement may increase the duration of the procedure and may require significant tissue dissection and muscle retraction. A further risk is that misplaced screws may cause neural and/or vascular injury, may result in excessive blood loss, increasing the need for transfusions, and may delay post-surgical healing. Healing around the screws that have been misplaced may result in poor outcomes such as prolonged recovery time, incomplete return to work, and excess rigidity leading to adjacent segmental disease requiring further fusions and re-operations.

Thus, there is a need for an improved interbody device that provides improved loadbearing surfaces and allows for contact of the interbody device with the stronger cortical bony rim of the vertebral endplate and that can be deployed using minimally invasive surgical (MIS) techniques. Moreover, there is also a need for an improved interbody device that is fixed to at least one of the adjacent vertebra such that the interbody spacer does not migrate postoperatively.

### SUMMARY

The present disclosure provides an expandable interbody device for proper spacing and fusion of two adjacent vertebrae. Embodiments of the present disclosure may include interbody devices for transforaminal lumbar interbody fusion (TLIF) or posterior lumbar interbody fusion (PLIF). The interbody device includes a cage having an anterior spacer, posterior spacer, a first lateral spacer, a second lateral spacer, and an actuator. The anterior spacer is rotatably coupled to the first lateral spacer by a first anterior hinge member and the anterior spacer is rotatably coupled to the second lateral spacer by a second anterior hinge member. The posterior spacer is rotatably coupled to the first lateral spacer by a first posterior hinge member and the posterior spacer is rotatably coupled to the second lateral spacer by a second posterior hinge member. The actuator is operatively coupled to the anterior spacer and the posterior spacer such that rotation of the actuator in a first direction causes the anterior spacer to travel along the actuator toward the posterior spacer such that the first and second lateral spacers move toward each other thereby expanding the cage from a collapsed configuration to an expanded configuration.

According to one embodiment, in a collapsed configuration, the interbody device has a cross section small enough to be deployed between adjacent vertebrae using MIS techniques such as through a standard endoscopic port or working tube and into the space between the vertebrae. According to one embodiment, the interbody device in this collapsed configuration is small enough to be deployed along pathways that minimize injury to nerves, muscles, and blood vessels, such as through Kambin's triangle. Once the device is successfully inserted into the intervertebral space, the actuator is rotated in the first direction to expand the cage of the device such that at least one of the spacers are positioned adjacent the cortical bony rims of the end plates of the adjacent vertebral bodies.

According to a further embodiment, one or more of the anterior and posterior spacers include openings to allow bone screws to pass through the spacer. According to one embodiment, a screw plate is provided that can be deployed using MIS techniques to engage with the device. The screw plate includes openings to allow bone screws to pass through the plate. According to one embodiment of the disclosure, once the interbody device has been deployed between vertebrae during a surgical procedure, the screw plate is inserted through the laparoscopic port and joined with the interbody device. Bone screws are then inserted through the openings in the screw plate and interbody device and into one or more of the vertebrae adjacent the interbody spacer to fix the interbody device in place. According to a further embodiment, the screw plate includes a protrusion or other structure that engages the actuator to prevent further movement of the actuator with respect to the interbody device to fix the configuration of the device in the expanded configuration.

According to some embodiments, the interbody device includes a cage having an anterior spacer, posterior spacer, a first lateral spacer, and a second lateral spacer. The anterior spacer is rotatably coupled to the first lateral spacer by a first anterior hinge member and the anterior spacer is rotatably coupled to the second lateral spacer by a second anterior hinge member. The posterior spacer is rotatably coupled to the first lateral spacer by a first posterior hinge member and the posterior spacer is rotatably coupled to the second lateral spacer by a second posterior hinge member. Once the interbody device is in place within the intervertebral space, the various spacers are manually manipulated by a surgeon to expand the cage and position the spacers in the desired location.

According to a further embodiment, anterior and posterior spacers include coupling features that allows a bone staple to be coupled with the cage once the bone staple is driven into the vertebrae adjacent the intervertebral space. The coupling feature of the anterior spacer includes a receiving channel having a retention feature. The coupling feature of the posterior spacer includes a receiving channel therethrough for receiving a portion of the bone staple.

According to one embodiment, the interbody device includes a cage having an anterior spacer, posterior spacer, a first lateral spacer, a second lateral spacer, and a central hub. The anterior spacer is rotatably coupled to the central hub by a first hinge member. The posterior spacer is rotatably coupled to the central hub by a second hinge member. The first lateral spacer is rotatably coupled to the central hub by a third hinge member. The second lateral spacer is rotatably coupled to the central hub by a fourth hinge member.

According to a further embodiment, the hinge members are extendable such that the length of the hinge members may be increased and/or decreased to allow the surgeon to position the spacers in the desired position within the intervertebral space.

According to some embodiments, posterior spacer includes a coupling feature that allows a bone staple to be coupled with the cage once the bone staple is driven into the vertebrae adjacent the intervertebral space. The coupling feature of the posterior spacer is a threaded bore dimensioned and configured to engage a mechanical fastener that couples the bone staple with the posterior spacer.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein:

FIG. 1A is an exploded view of an interbody device according to an embodiment of the disclosure;

FIG. 1B is a front perspective view of an interbody device in a collapsed configuration according to an embodiment of the disclosure;

FIG. 2 is a top view of the interbody device of FIG. 1B in an expanded or partially expanded configuration;

FIG. 3 is another top view of the interbody device of FIG. 1B;

FIG. 4 is a side view of the interbody device of FIG. 1B;

FIG. 5A is a diagram of a portion of the spinal column illustrating Kambin's triangle;

FIG. 5B is a diagram showing the insertion of the interbody device of FIG. 1B through a working tube according to an embodiment of the disclosure;

FIG. 6A is a top view of the interbody device of FIG. 1B in a collapsed configuration placed within the intervertebral space according to an embodiment of the disclosure;

FIG. 6B is a top view of the interbody device of FIG. 1B in an expanded configuration within the intervertebral space according to an embodiment of the disclosure;

FIG. 7 is a rear view of an anterior spacer of the interbody device of FIG. 1B;

FIG. 8 is a front view of a posterior spacer of the interbody device of FIG. 1B;

FIG. 9 is a medial side view of a first lateral spacer of the interbody device of FIG. 1B;

FIG. 10 is a medial side view of a second lateral spacer of the interbody device of FIG. 1B;

FIG. 11 is a cross-sectional view along line 11-11 of FIG. 3;

FIG. 12 is a cross-sectional view along line 12-12 of FIG. 3;

FIG. 13 is a cross-sectional view along line 13-13 of FIG. 3;

FIG. 14 is a cross-sectional view along line 14-14 of FIG. 3;

FIG. 15 is an exploded view of the interbody device of FIG. 1B according to an embodiment of the disclosure;

FIG. 16 is a front perspective view of an interbody device with a screw plate in a collapsed configuration according to another embodiment of the disclosure;

FIG. 17 is a side view of the interbody device and screw plate of FIG. 16;

FIG. 18A is a top view of the screw plate of FIG. 16 separate from the interbody device;

FIG. 18B is a side view of the screw plate of FIG. 16;

FIG. 18C is a side view of a posterior spacer according to an embodiment of the disclosure;

FIG. 19 is an exploded view of an interbody device and screw plate according to another embodiment of the disclosure;

FIG. 20 is a front perspective view of an interbody device in a collapsed configuration according to an embodiment of the disclosure;

FIG. 21 is a front perspective view of a cage of an interbody device according to an embodiment of the disclosure in a collapsed configuration;

FIG. 22 is a rear view of an anterior spacer of the interbody device of FIG. 21;

FIG. 23 is a front view of a posterior spacer of the interbody device of FIG. 21;

FIG. 24 is a side view of a bone staple according to an embodiment of the disclosure;

FIG. 25A is an enlarged view of the bone piercing member of FIG. 24 according to one embodiment of the disclosure;

FIG. 25B is an enlarged view of the bone piercing member of FIG. 24 according to another embodiment of the disclosure;

FIG. 26 is an exploded view of an interbody device according to an embodiment of the disclosure engaging the bone piercing member of FIG. 24;

FIG. 27 is a rear perspective view of the interbody device and bone piercing member of FIG. 26;

FIG. 28 is a top view of an interbody device according to an embodiment of the disclosure;

FIG. 29 is a top view of the interbody device shown in FIG. 28 in an extended or partially extended configuration;

FIG. 30A is a rear view of an anterior spacer of the interbody device of FIG. 28;

FIG. 30B is a front view of a posterior spacer of the interbody device of FIG. 28;

FIG. 31A is a side view of a lateral spacer of the interbody device of FIG. 28;

FIG. 31B is a side view of a lateral spacer of the interbody device of FIG. 28;

FIG. 32 is a front perspective view of a central hub of the interbody device of FIG. 28;

FIG. 33 is a top view of a hinge member of the interbody device of FIG. 28;

FIG. 34 is a top view of the central hub illustrating the coupling of a hinge member and the central hub;

FIG. 35A is a locking mechanism according to a first aspect in a first position;

FIG. 35B is the locking mechanism of FIG. 35a in a second position;

FIG. 35C is a cross section of a segment of a hinge member according to an embodiment of the disclosure; and

FIG. 36 an exploded view of an interbody device according to an embodiment of the disclosure.

### DETAILED DESCRIPTION

Exemplary embodiments of the disclosure will now be described below by reference to the attached Figures. The described exemplary embodiments are intended to assist the understanding of the invention and are not intended to limit the scope of the invention in any way. Like reference numerals refer to the elements throughout.

For the purposes of this disclosure, the terms “distal,” “distal of” and the like will be used throughout this disclosure to refer to the direction or relative position away from the surgeon inserting the device and toward the body of a patient being treated using the device. The terms “proximal,” “proximally,” “proximal of” and the like will be used throughout this disclosure to refer to the direction toward the surgeon inserting the device and away from the body of the patient being treated using the device. The terms “medial,” “medially,” “medial of” and the like will be used throughout this disclosure to refer to the direction toward the midline of the body of the patient. The terms “lateral,” “laterally,” “lateral of” and the like will be used throughout this disclosure to refer to the direction away from the midline of the body of the patient. The terms “superior,” “superiorly,” “superior of,” “cranial” and the like will be used throughout this disclosure to refer to the direction toward the head of the patient relative to a transverse plane of the patient. The terms “inferior,” “inferiorly,” “inferior of,” “caudal” and the like will be used throughout the disclosure to refer to the direction away from the head of the patient relative to a transverse plane of the patient. The terms “anterior,” “anteriorly,” “anterior of” and the like will be used throughout the disclosure to refer to the direction toward the front of the body of the patient relative to a frontal plane of the patient. The terms “posteriorly,” “posterior of,” and the like will be used throughout the disclosure to refer to the direction toward the back of the body of the patient relative to the frontal plane of the patient.

A spinal fusion is typically employed to eliminate pain caused by a degenerated or diseased disc. Spinal fusion is a procedure whereby a diseased disc is surgically removed and the endplates of adjacent vertebrae are caused to grow together to form an immobile, stable structure. According to one embodiment of the disclosure, the disc is removed and an interbody device is inserted into the intervertebral disc space between the endplates formerly occupied by the disc. Upon successful fusion, the interbody device becomes permanently fixed within the intervertebral disc space. In some embodiments, an interbody device may be designed to be inserted into the intervertebral space using minimally invasive surgery (MIS). A device according to the disclosure may be used as part of a number of surgical procedures, including but not limited to, Transforaminal Lumbar Interbody Fusion (TLIF) and Posterior Lumbar Interbody Fusion (PLIF).

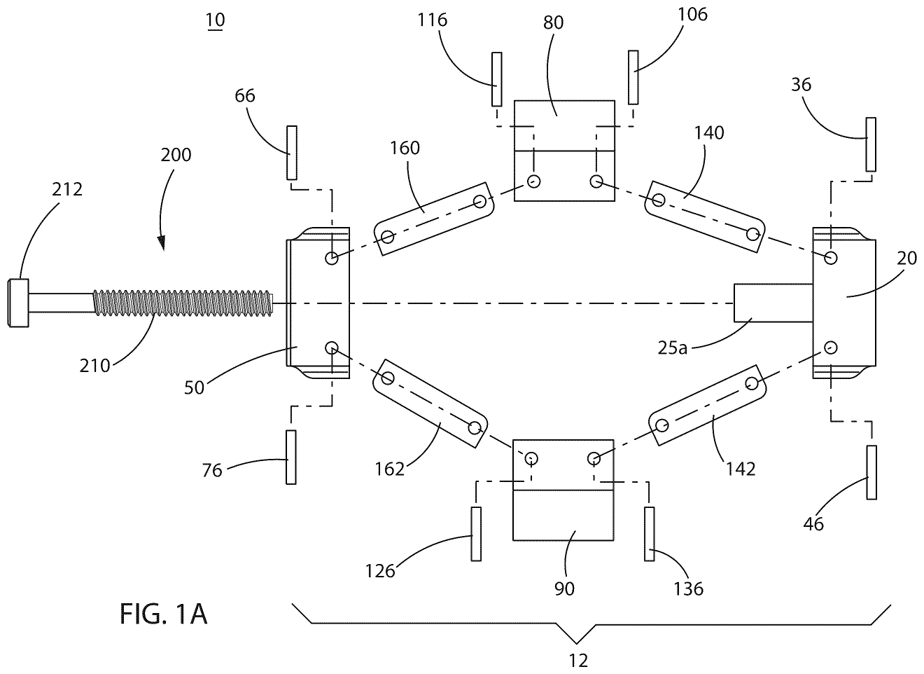

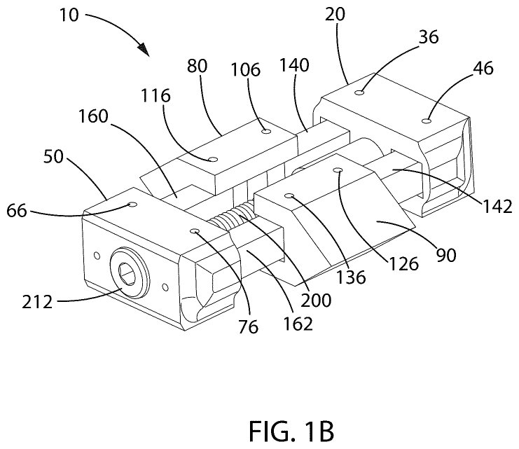

Referring to FIGS. 1A and 1B, an exemplary embodiment of an interbody device 10 according to the present disclosure is shown. Interbody device 10 includes cage 12 having an anterior spacer 20, a posterior spacer 50, lateral spacers 80, 90, hinge members 140, 160, 142, and 162 and actuator 200. In an exemplary embodiment, radiographic markers are embedded within one or more components of cage 12. Aactuator 200 extends through posterior spacer 50 and connects with anterior spacer 20. Hinge member 142 connects anterior spacer 20 with lateral spacer 90. Hinge member 162 connects posterior spacer 50 with lateral spacer 90. Hinge member 140 connects anterior spacer 20 with lateral spacer 80. Hinge member 160 connects posterior spacer 50 with lateral spacer 80. As will be explained below, connections between the hinge members and spacers allow the hinge members to rotate with respect to the spacers.

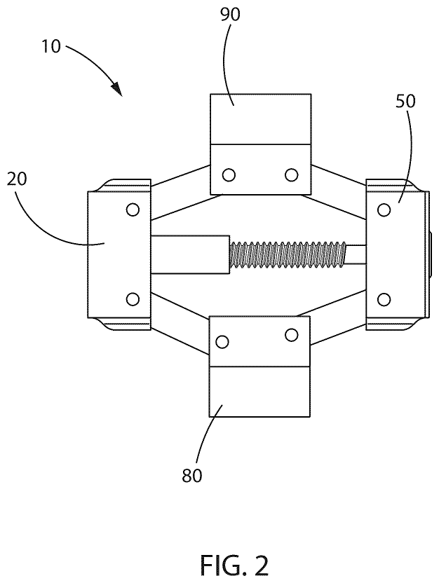

Actuator 200 extends through posterior spacer 50. Head 212 of actuator 200 rests against a posterior surface of posterior spacer 50. At least a portion of actuator 200 is threaded. The thread engages with threads formed on anterior spacer 20. Rotation of head 212 with respect to device 10 in one direction pulls anterior spacer 20 toward posterior spacer 50. This causes hinge members 162, 142, 140, 160 to rotate about their connection with spacers 20, 50, 80, 90. As shown in FIG. 1B, when actuator 200 is rotated in one direction, anterior and posterior spacers 20, 50 are at a maximum distance from one another, causing device 10 to be in the collapsed configuration. Rotation of actuator 200 in a second direction, as shown in FIG. 2, anterior and posterior spacers 20, 50 are drawn toward each other, putting interbody spacer 10 in an expanded or partially expanded configuration. In the expanded configuration, spacers and hinge members form an enlarged opening 18m as shown in FIG. 2. According to one embodiment, bone growth material may be introduced into space 18 to promote fusion of adjacent vertebrae, as will be discussed below.

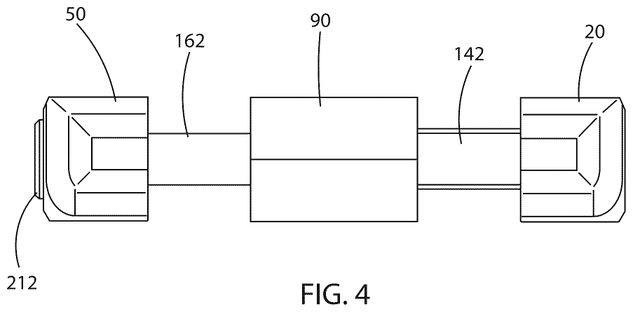

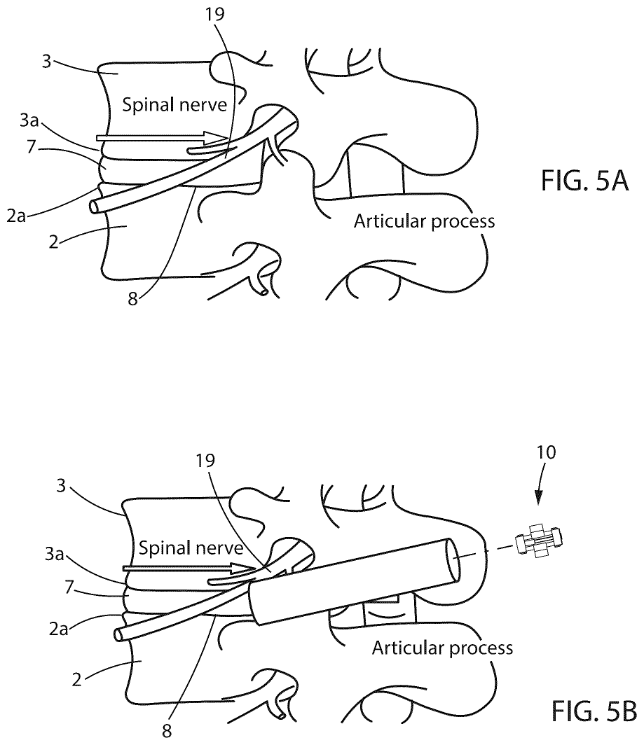

According to one embodiment, when device 10 is in the collapsed configuration, the device has a sufficiently small cross section to be introduced into an intervertebral space using MIS techniques. According to a further embodiment, by providing the device in the collapsed configuration, access can be made vis Kambin's triangle, an anatomical region adjacent the disc space. Accessing the disc space through this route may reduce the need to resect or displace spinal nerves, muscles and blood vessels. FIG. 5A is a schematic view of Kambin's triangle. Kambin's triangle 8 provides a corridor for posterolateral access for spinal surgery. Kambin's triangle 8 can be defined as a right triangle over the intervertebral disc 7 viewed dorsolaterally. The hypotenuse is defined by the exiting nerve 9, the base is the superior border of the inferior vertebra 2, and the height is the traversing nerve root (not shown).

According to one embodiment, interbody 10 is inserted into the intervertebral disc space through Kambin's triangle while in a collapsed configuration. That is, the cross section of device 10 in the collapsed configuration is small enough to pass through Kambin's triangle whose height and width, respectively, averages about 12 mm and 10 mm (L1-L2), 13 mm and 11 mm (L2-L3), 17 mm and 11 mm (L3-L4), and 18 mm and 12 mm (L4-L5). According to one embodiment, the respective width of the interbody device 10 in a collapsed configuration is between about 5 mm and about 15 mm. According to a preferred embodiment the width in a collapsed configuration is less than about 10 mm.

As previously stated, surgical techniques contemplated by the present disclosure include, for example, MIS and open surgery. For case of description, this disclosure will refer to an MIS technique, although other surgical techniques known to one of ordinary skill in the art will be appreciated. As seen in FIG. 5B, in an exemplary embodiment, a surgical incision is made and a working tube 19 is surgically placed through Kambin's triangle to allow access to the intervertebral space adjacent the diseased disc. The disc is removed using instruments deployed through working tube 19 using techniques known in the field of the disclosure. Interbody spacer 10 is then delivered through the working tube and into the intervertebral space. In some circumstances, approaching the disc via Kambin's triangle may reduce the risk of damage to the surrounding nerves, vasculature, and muscles and cause less trauma to the patient.

The endoscope or working tube 19 may have a circular cross section, oval cross section, square cross section, or rectangular cross section. Though it is envisioned, any endoscope or working tube that is known in the art may be used, such as a C-shaped cannula or open square cannula. Preferably, the endoscope or working tube 9 is at least 11 mm at the narrowest point. However, it is envisioned that any size or shape endoscope and/or working tube may be used so long as it is sized and dimensioned to allow the interbody spacer 10 in its collapsed configuration to be inserted therethrough and into the intervertebral space. In some exemplary embodiments, endoscopic foraminoplasty may be performed to enlarge the route that provides access to the intervertebral disc space such that the interbody spacer 10, endoscope, working tube 9, and/or surgical instruments have easier access to the intervertebral disc space as dictated by the patient's anatomy.

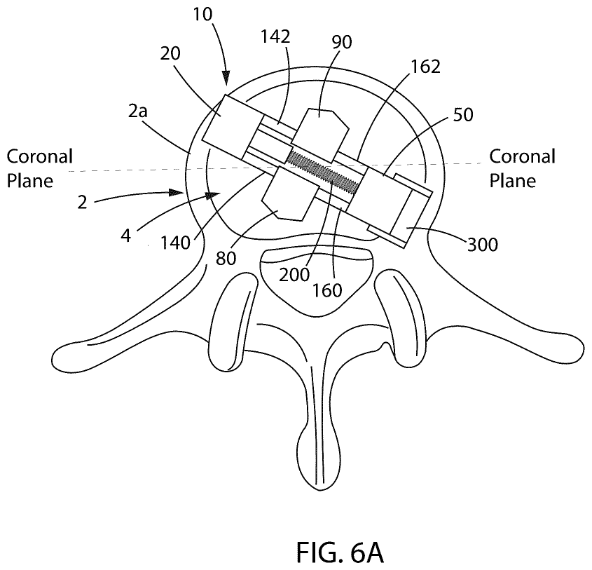

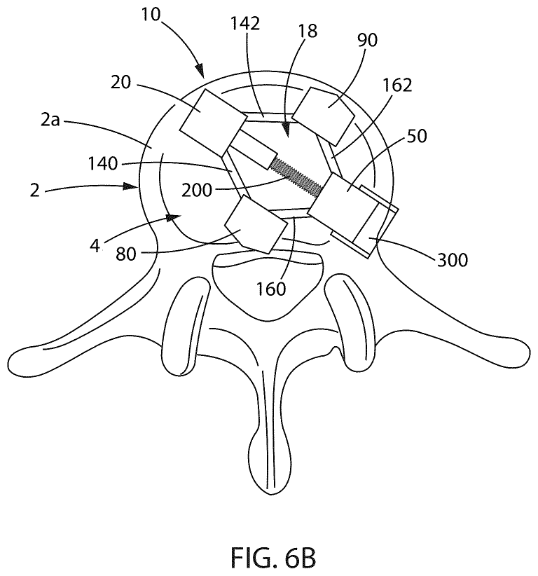

Referring now to FIGS. 6A and 6B, an exemplary embodiment of interbody device 10 according to the present disclosure is shown inserted into the intervertebral disc space. In an exemplary embodiment, interbody device 10 is configured to be inserted into the disc space in a first collapsed configuration at an angle, preferably about 45 degrees, relative of the coronal plane. However, the anatomy of the patient and the selected surgical technique will dictate the angle of insertion. According to some embodiments, interbody device 10 may be inserted at an angle ranging from 30 about degrees to about 60 degrees relative to the coronal plane.

Upon being placed in a desired location within the disc space, the expandable interbody spacer is expanded from the collapsed configuration in FIG. 6A to the expanded configuration as shown in FIG. 6B. According to one embodiment, actuator 200 is operated to move the anterior spacer 20 toward the posterior spacer 50, thereby reducing the length of the device and increasing its width. As shown in the embodiment of FIG. 6B, interbody device 10 may be adjusted so that one or more of the spacers 20, 50, 80, 90 engage the endplates 4 and 5 of the adjacent vertebral bodies 2 and 3 and contact the cortical rim 2a, 3a of the adjacent vertebrae. According to one embodiment, in the expanded configuration, device 10 maintains normal intervertebral disc spacing and restores spinal stability, thereby facilitating intervertebral fusion. The expanded configuration encompasses full or partial expansion of device 10 so that the spacers are positioned properly with respect to the cortical bony rim of the vertebral endplate.

As will be discussed in greater detail below, the desired height and/or angle of lordosis may be selected by choosing a configuration of interbody device 10 having the desired characteristics to achieve the desired height and/or angle of lordosis. According to one embodiment, the size and shape of spacers 20, 50, 80, 90 are selected to provide a selected orientation of adjacent vertebrae. According to one embodiment, device 10 is provided as a kit including a selection of components, such as the spacers 20, 50, 80, 90 that can be assembled at the time of use prior to insertion of the interbody device.

In the expanded configuration, shown in FIG. 6B, opening 18 may be adapted to hold a quantity of cadaver bone graft or similar bone growth inducing material between the adjacent vertebrae. In an exemplary embodiment, bone graft or similar bone growth inducing material can be introduced around and/or within the interbody device 10 to promote and facilitate intervertebral fusion. As a non-limiting example, once the interbody device is in place within the intervertebral space, bone graft or similar bone growth inducing material is introduced around and/or within the interbody device 10 through an insertion tool.

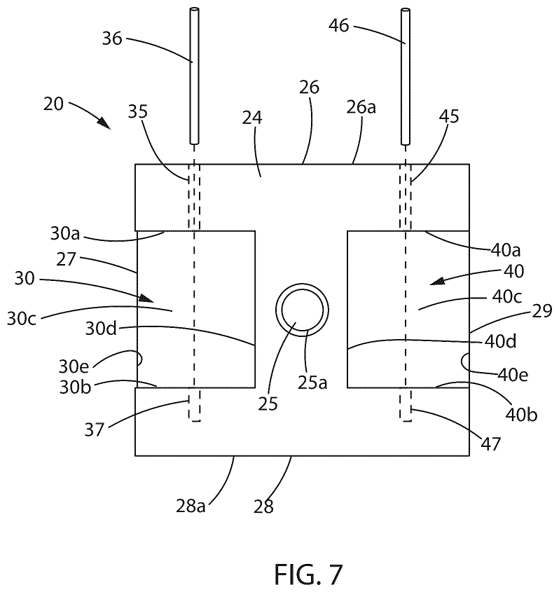

FIG. 7 shows the proximal facing side of anterior spacer 20. Anterior spacer 20 has a front wall 22, a rear wall 24, a top wall 26, a bottom wall 28, side walls 27, 29 and hinge member receiving channels 30, 40. The front wall 22, rear wall 24, and side walls 27 and 29 define the height of the anterior spacer 20. At least a portion of the top wall 26 and bottom wall 28 include a contact surface 26a and 28a. Contact surface 26a of the top wall 26 is configured to contact the inferior end plate of the adjacent vertebral body located superiorly to the vertebral space the interbody spacer 10 resides in, as shown, for example, in FIG. 6B. Likewise, the contact surface 28a of the bottom wall 28 is configured to contact the superior end plate of the adjacent vertebral body located inferiorly to the vertebral space the interbody spacer 10 resides in.

Anterior spacer 20 forms the distal end of interbody device 10. According to one embodiment, contact surfaces 26a, 28a of anterior spacer 20 are rectangular. However, it is envisioned that any suitable shape may be used so long as sufficient contact is made between contact surface 26a and the corresponding inferior vertebral endplate and contact surface 28a and the corresponding superior endplate. In the embodiment shown, the contact surfaces 26a and 28a are substantially planar and parallel with one another. In another embodiment, the contact surfaces 26a and/or 28a are sloped to accommodate the natural anatomic relationship between adjacent vertebral bones and maintain the normal lordotic curvature of the spine. Likewise, the height of anterior spacer 20, that is, the distance between contact surfaces 26a, 28a, may be selected to achieve a desired height and/or lordosis.

The contact surfaces 26a and 28a may be coated with any suitable material, such as hydroxyl apatite, beta-tricalcium phosphate, anodic plasma chemical treated titanium, or other similar coatings that improve osseointegration of the interbody spacer 10. Furthermore, contact surfaces 26a and 28a may also include raised surface features or textures adapted to promote bone growth. It should be further understood that surface features could be formed on any side of the anterior spacer 20 to enhance bone growth on the interbody spacer 10 and/or within the opening 18 of the interbody spacer 10.

As shown in FIGS. 1A and 1B, two hinge members 140 and 142 connect anterior spacer 20 with lateral spacers 80, 90. As shown in FIG. 7, anterior spacer 20 includes hinge member receiving channels 30, 40. Channels 30, 40 are sized to each receive one end of hinge members 140, 142. According to one embodiment, channels 30, 40 are each defined by a top wall 30a, 40a, bottom wall 30b, 40b, front wall 30c, 40c, side wall 30d, 40d, and side wall 30c, 40c. The top wall 26 of the anterior spacer 20 has apertures 35, 45 that are dimensioned and configured to receive retaining pins 36, 46. Bottom walls 30b, 40b of each have a bore 37, 47 that is coaxially aligned with the respective aperture 35, 45 and is configured and dimensioned to receive at least a portion of the respective retaining pin 36, 46. It should be appreciated that apertures 35, 45 and bores 37, 47 may be inversely related in the sense that the aperture may be locate through either the top or the bottom surface of anterior spacer 20.

Rear wall 24 of the anterior spacer 20 includes a threaded bore 25 that is generally centrally located. Bore 25 is configured and dimensioned to receive an actuator shaft 210 of actuator 200 that will be discussed in greater detail below. According to one embodiment, a threaded sleeve 25a is aligned with the bore 25 and extends from the rear wall 24 such that bore 25 is also extended proximally, as shown in FIG. 1A. Sleeve 25a may be monolithically formed with anterior spacer 20 or may be welded or otherwise fixed to rear wall 24 of anterior spacer 20. In such an embodiment, the threading of the bore 25 is continuous with the threading of the sleeve 25a. As will be described in more detail below, this allows the anterior spacer 20 a greater length of travel along the actuator shaft 210 as the actuator 200 is rotated about an axis and engages the threads of bore 25.

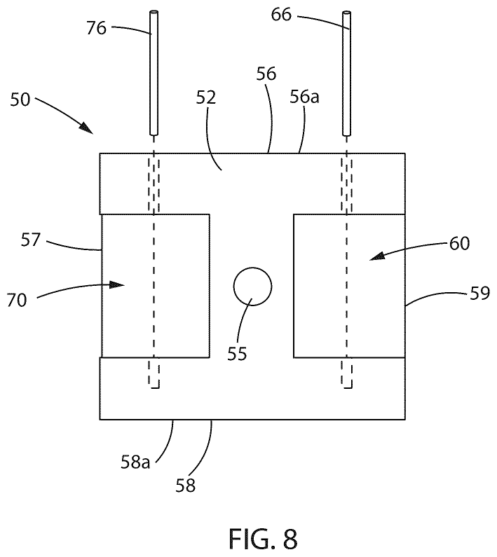

FIG. 8 shows the distal facing side of posterior spacer 50. Posterior spacer 50 defines two hinge member receiving channels 60, 70, similar to receiving channels 30, 40 discussed above with respect to the anterior spacer 20. As shown in FIG. 1A, hinge members 160, 162 join posterior spacer 50 with lateral spacers 80, 90. Pins 66, 76 rotatably connect hinge members 160, 162 with posterior spacer 50 in a manner similar to the connection with anterior spacer 20, described above.

At least a portion of the top wall 56 and bottom wall 58 include a contact surface 56a and 58a. Contact surfaces 56a and 58a are substantially similar as contact surfaces 26a and 28a of anterior spacer 20.

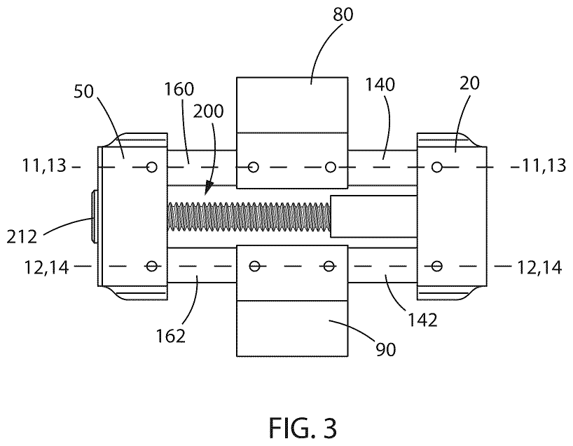

Posterior spacer 50 includes an aperture 55 that is generally centrally located and extending through the spacer from the posterior to the anterior surface thereof. As shown in FIG. 3, aperture 55 extends through posterior spacer 50. Aperture 55 is dimensioned and configured to receive actuator shaft 210 of the actuator 200. In the embodiment shown, aperture 55 is smooth. According to one embodiment, actuator shaft 210 is threaded such that the distal portion of actuator shaft is engaged with threads of bore 25 and/or sleeve 25a so that rotation of shaft 210 pulls anterior and posterior spacers 20, 50 toward one another.

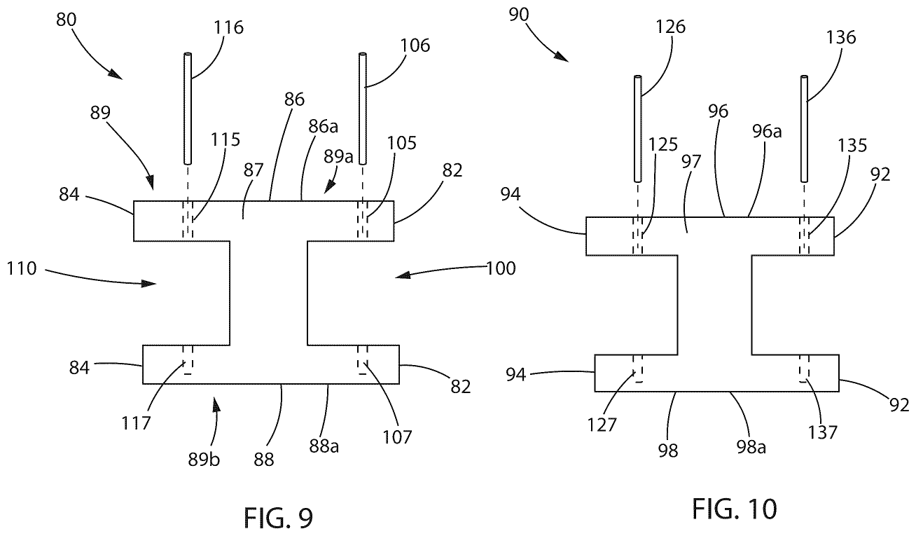

FIG. 9 shows lateral spacer 80 viewed from within space 18 looking outward. Lateral spacer 80 has a front wall 82, a rear wall 84, a top wall 86, a bottom wall 88, a side wall 87 and at least two angled surfaces 89. Angled surfaces 89a, 89b are configured to distract the adjacent vertebral bodies when the interbody device 10 is expanded into an intervertebral space. In another exemplary embodiment, lateral spacer 80 has only one angled surface to distract the adjacent vertebral bodies when the interbody spacer 10 is expanded into an intervertebral space.

The front wall 82, rear wall 84, and side wall 87 define the height of the lateral spacer 80. At least a portion of the top wall 86 and bottom wall 88 include a contact surface 86a and 88a. Contact surfaces 86a and 88a are similar to contact surfaces 26a and 28a of the anterior spacer 20. The side wall 87 of lateral spacer 80 has two hinge member receiving channels 100, 110. Hinge member receiving channels 100, 110 are substantially similar to hinge member receiving channels 30, 40 of anterior spacer 20. Lateral spacer 80 is joined with hinge member 140, 160 by pins 106, 116 that extend through apertures 105 and 115, respectively, and are received in apertures 107, 117.

FIG. 10 shows lateral spacer 90 viewed from within space 18 looking outward. Lateral spacer 90 is joined with hinge member 142, 162 by pins 126, 136 that extend through apertures 125 and 135, respectively, and are received in apertures 127, 137 in a manner similar to lateral spacer 80, described above.

According to one embodiment, hinge members 140, 142, 160, 162 have a square cross-section. However, it should be understood that the anterior hinge members may have any suitable cross section shape so long as the shape compliments the shape of the corresponding hinge member receiving channels.

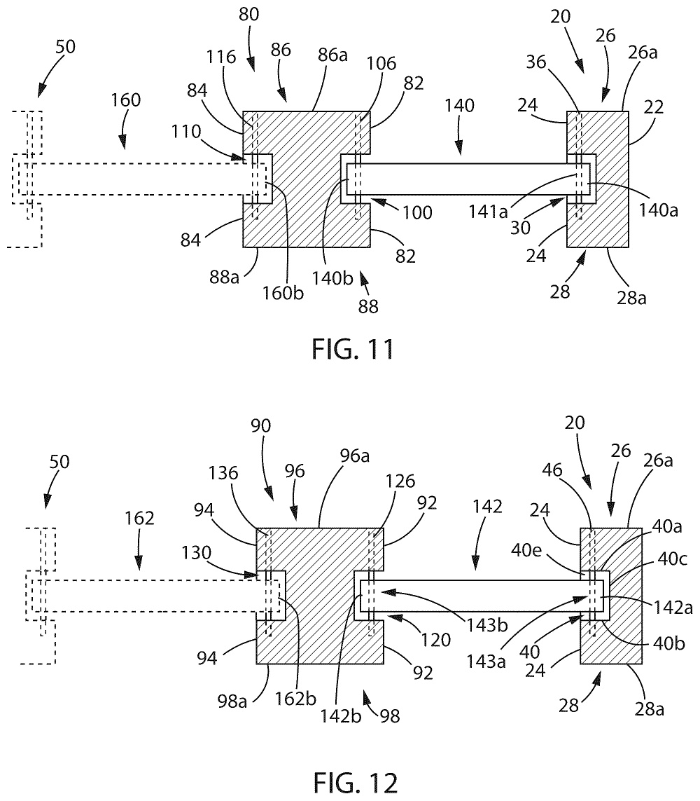

FIGS. 11 and 12 are partial cross-sectional views along planes identified in FIG. 3 showing the connection between lateral spacers 80, 90 and anterior spacer 20. As shown in FIG. 11, first anterior hinge member 140 has a first end 140a and a second end 140b. The first end 140a of the anterior hinge member 140 is dimensioned and configured to be received by the hinge member receiving channel 30 of the anterior spacer 20. First end 140a of the anterior hinge member 140 has an aperture 141a that is configured and dimensioned to receive pin 36 such that the hinge member 140 is rotatably connected in the hinge member receiving channel 30. The second end 140b of the anterior hinge member 140 is dimensioned and configured to be received by the hinge member receiving channel 100 of lateral spacer 80. Likewise, second end 140b of first anterior hinge member 140 has an aperture 141b that is configured and dimensioned to receive pin 106 such that the first anterior hinge member 140 is rotatably connected in the hinge member receiving channel 100 of lateral spacer 80.

As shown in FIG. 12, second anterior hinge member 142 has a first end 142a and a second end 142b. First end 142a of the anterior hinge member 142 is dimensioned and configured to be received by the hinge member receiving channel 40 of the anterior spacer 20. First end 142a of the anterior hinge member 142 has an aperture 143a that is configured and dimensioned to receive pin 46 such that the second anterior hinge member 142 is rotatably connected in the hinge member receiving channel 40. Second end 142b of anterior hinge member 142 is dimensioned and configured to be received by the hinge member receiving channel 120 of the second lateral spacer 90. Likewise, second end 142b of second anterior hinge member 142 has an aperture 143b that is configured and dimensioned to receive pin 106 such that the second anterior hinge member 142 is rotatably connected in the hinge member receiving channel 120 of the second lateral spacer 90.

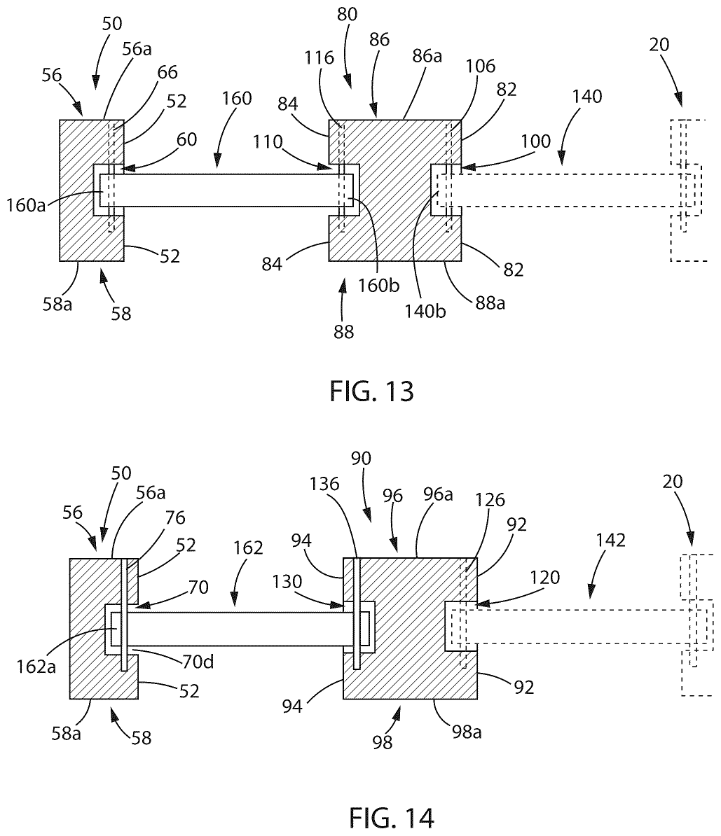

FIGS. 13 and 14 are partial cross-sectional views along planes identified in FIG. 3 showing the connection between lateral spacers 80, 90 and posterior spacer 50. Hinge member 160 is rotatably connected to posterior spacer 50 and lateral spacer 80 by pins 66, 116 substantially similar to the connection between anterior spacer 20 and lateral spacer 80, as described above. Likewise, as shown in FIG. 14 posterior hinge member 162 is rotatably connected to posterior spacer 50 and lateral spacer 90 by pins 76, 136 substantially similar to the connection between anterior spacer 20 and lateral spacer 90, as described above.

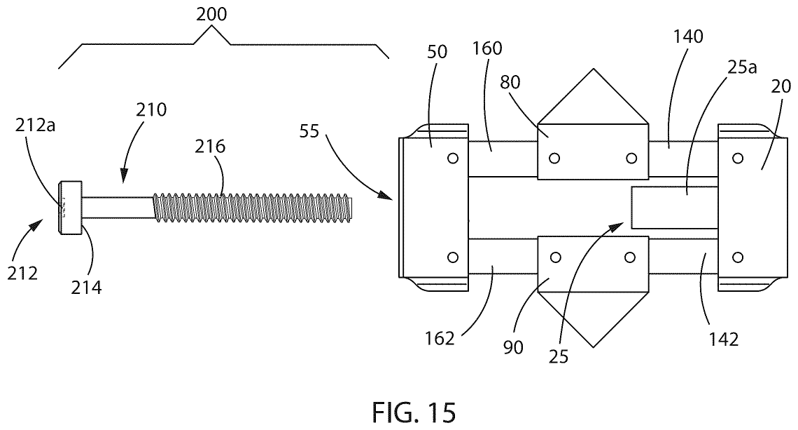

Referring now to FIG. 15, actuator 200 includes an actuator shaft 210 that extends along a central axis that is oriented along a longitudinal direction. In the exemplary embodiment shown, the actuator shaft 210 is an elongated bolt having a head portion 212. Shoulder 214 contacts a posterior-facing surface of spacer 50 adjacent to aperture 55. Threaded portion 216 engages the threads of the sleeve 25a and/or bore 25 of the anterior spacer 20. The head portion 212 of shaft 210 includes a mating feature or drive 212a for engaging a driving tool. The mating feature or drive 212a of the head portion 212 is configured to receive the tip of a driving tool, for example, by a hexagonal opening sized to accept a hex driver. Other shapes for drive 212a may be used within the scope of the disclosure. According to one embodiment, opening 55 may include a countersink portion to recess or partially recess head portion 212 below the posterior surface of posterior spacer 50. According to this embodiment, shoulder 214 of the actuator shaft 210 is configured and dimensioned to fit within the countersunk portion of aperture 55 so that shaft 210 can freely rotate relative to the posterior spacer 50. The threading on the outer surface of the threaded portion 216 of the of the actuator shaft 210 is complementary to the threading of the threaded bore 25 and/or sleeve 25a of the anterior spacer 20. The threading of the threaded portion 216 may be a single-lead threading or a multi-lead threading, e.g., double-lead, triple-lead or quadruple-lead threading, as is known in the field of the disclosure.

As the actuator 200 is rotated in a first direction, the threaded portion 216 of the actuator shaft 210 engages the complimentary threads of the threaded bore 25 of the anterior spacer 20 so that the anterior spacer travels along the actuator shaft toward the posterior spacer 50, thereby causing the lateral spacers 80, 90 to move away from one another such that the interbody spacer 10 expands, as shown in FIG. 1B. Likewise, as the actuator 200 is rotated in a second direction, the threaded portion 216 of the actuator shaft 210 engages the complimentary threads of the threaded bore 25 of the anterior spacer so that the anterior spacer travels along the actuator shaft away from the posterior spacer 50, thereby causing the lateral spacers 80, 90 to move toward one another such that the interbody device 10 moves toward a collapsed configuration.

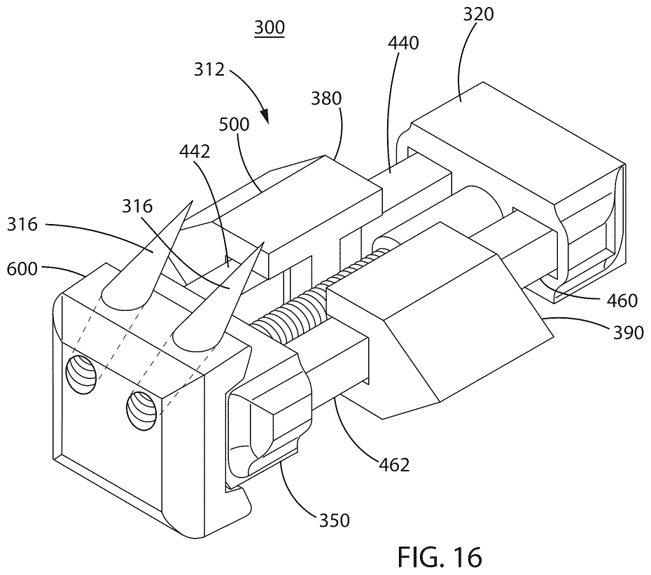

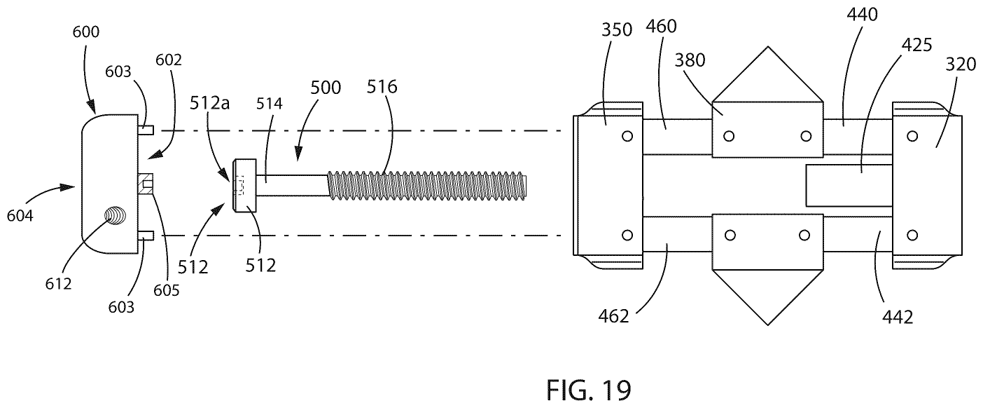

According to a further embodiment of the disclosure, a screw plate and bone screws may be provided to secure the device to one or more of the adjacent vertebrae. Referring now to FIGS. 16-19, an exemplary embodiment of an interbody device 300 according to the present disclosure is shown. Interbody device 300 includes cage 312 having an anterior spacer 320, a posterior spacer 350, lateral spacers 380, 390, hinge members 440, 460, 442, 462 and actuator 500. In an exemplary embodiment, radiographic markers are embedded within one or more components of cage 312. As discussed with respect to the previous embodiments, spacers 320, 350, 380, 390 are joined by hinge members 440, 460, 442, 462 so that motion of the anterior and posterior spaces by actuator 500 moves the device from a collapsed to an expanded configuration. Device 300 includes screw plate 600 that is connected with cage 312 to secure the device in the intervertebral space.

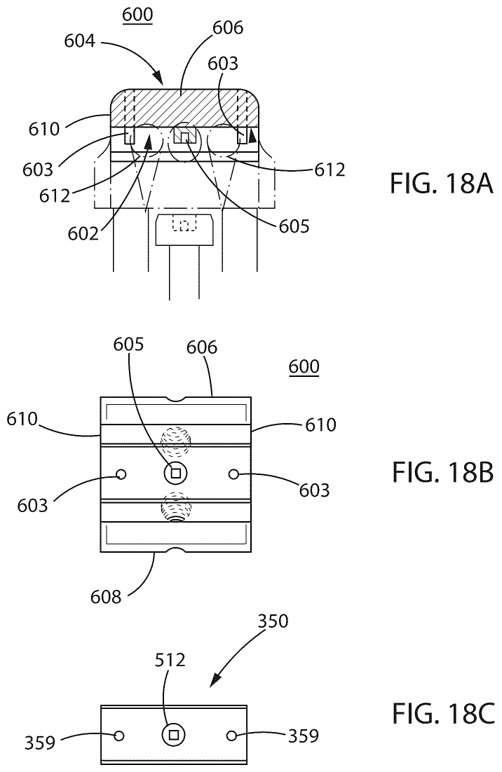

Screw plate 600 serves as an interface for the cage 312 and bone screws 316. As shown in FIGS. 18A and 18B, screw plate 600 includes an inner surface 602 and an outer surface 604 interconnected by a top end 606, bottom end 608 and sides 610. The screw plate 600 is configured for attachment to the cage 312 and, in particular, for attachment to the rear wall of the posterior spacer 350. The inner surface 602 of the screw plate 600 includes at least one pin 603 that is configured and dimensioned to be received by the apertures 359 of the posterior spacer 350. As shown in FIG. 18C, posterior spacer 350 includes one or more apertures 359 configured and dimensioned to receive pins 603 of the screw plate 600. In the embodiment shown, two apertures 359 are present for receiving pins 603 of the screw plate 600. Instead of bone screws, elements 316 may be pins, or other elongated inserts.

As shown in FIGS. 18A and 18B, inner surface 602 of screw plate 600 has a locking protrusion 605 extending away from the screw plate 600. FIG. 18C shows the posterior surface of posterior spacer 350 adopted to engage with screw plate 600. When screw plate 600 is engaged with posterior spacer 350, locking protrusion 605 engages with mating feature 512a of the head 512 of shaft 510. The locking protrusion 605 may be configured to mate with any suitable driving tool tip feature, such as, a compatible hex, flat, or Philips shapes that engage with actuator 500 to prevent the actuator from rotating, thus fixing cage 312 in an expanded configuration.

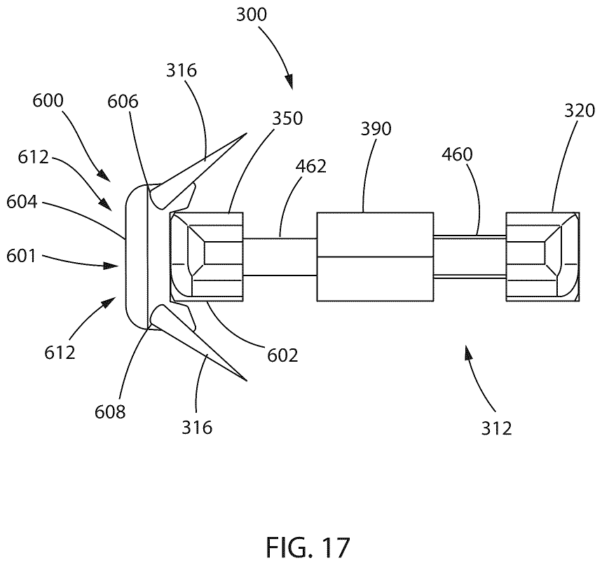

The outer surface 604 of the screw plate 600 includes one or more bone screw openings 612 sized and configured to receive bone screws 316 in a manner that permits the bone screws 316 to pass therethrough and engage with the superior or inferior vertebral body. As shown in FIG. 17, the bone screw openings 612 are at about a 45-degree angle with respect to the plane defined by spacers 320, 350, 380, 390. However, it is envisioned the screw openings 612 may be at any angle so long as the bone screws 316 are able to engage the superior or inferior vertebral body so that the interbody spacer 300 is secured within the disc space. It is also contemplated within this disclosure that bone screw openings 612 may be oblong such that the bone screws 316 may be inserted therethrough at varying angles.

One or more bone screw openings 612 are angled toward the top end 606 such that bone screws 316 inserted therein are directed into the side of the vertebral body located between the lower endplate and upper endplate of the adjacent upper vertebra. One or more bone screw openings 612 is angled toward the bottom end 608 such that bone screws 316 inserted therein are directed into the side of the vertebral body located between the lower endplate and upper endplate of the adjacent lower vertebra.

The components of interbody device 10, 300 described herein can be fabricated from biologically acceptable materials suitable for medical applications, including metals, synthetic polymers, ceramics and bone material and/or their composites. For example, the components of interbody spacer 10, individually or collectively, can be fabricated from materials such as stainless-steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL®), ceramic and composites thereof such as calcium phosphate (e.g., SKELITE™). It is contemplated within this disclosure that interbody device 10, 300 may be made of allograft or other bioabsorbable material.

Various components of interbody device 10 may be formed or constructed of material composites, including but not limited to the above-described materials, to achieve various desired characteristics such as strength, rigidity, elasticity, compliance, biomechanical performance, durability and radiolucency or imaging preference. The components of interbody device 10, individually or collectively, may also be fabricated from a heterogeneous material such as a combination of two or more of the above-described materials. The components of the interbody spacer 10 may be formed using a variety of subtractive and additive manufacturing techniques, including, but not limited to machining, milling, extruding, molding, 3D-printing, sintering, coating, vapor deposition, and laser/beam melting. Furthermore, various components of the interbody spacer may be coated or treated with a variety of additives or coatings to improve biocompatibility, bone growth promotion or other features. For example, the anterior spacer 20, 320, posterior spacer 50, 350 and lateral spacers 80, 90, 380, 390 may be selectively coated with bone growth promoting or bone ongrowth promoting surface treatments that may include, but are not limited to: titanium coatings (solid, porous or textured), hydroxyapatite coatings, or titanium plates (solid, porous or textured).

According to some embodiments, cage 12, 312 has a cage height defined by the height of the spacers 20, 50, 80, 90, 320, 350, 380, 390 between about 6 and about 16 mm. In a collapsed state, the lateral dimension of cage 12, 312 (that is, traverse to the path that the device would travel through a working tube 9 used in an MIS procedure) is =may be less than about 10 mm. In an expanded state, the lateral dimension of cage 12, 312 may be adjusted to between about 6 mm and about 22 mm.

### The Surgical Method

Exemplary surgical steps for practicing one or more of the foregoing embodiments will now be described.

The patient is placed in a prone position on a surgical table, typically a Jackson table, and the operative field is disinfected and draped. Using preoperative fluoroscopy, the location of the targeted segmental space is marked on the patient's skin and a stab incision is made. A guide needle is then inserted into the desired location, which may be determined by C-arm fluoroscopy. The initial incision is then extended longitudinally to a length of about 10-30 mm.

The surgeon then uses the enlarged incision and guide needle to guide an initial dilator that separates muscle fibers and provides access to the underlying spine without cutting through the muscles. Once the initial dilator is docked on the back of the spine, a series of progressively larger dilators are added gradually increasing the diameter of the opening until enough room for the addition of an endoscope and/or working tube 9 has been achieved. It should be noted, to limit and/or reduce any drilling needed later on in the procedure, before the introduction of the next size dilator, the surgeon may decide to guide a reamer of substantially similar size of the current dilator in place to enlarge the foramen.

The surgeon then determines if the surgical corridor to be used is adequately sized or needs to be enlarged. This may be done by first determining the size of the interbody spacer 10, 300 needed to produce the desired outcome for the patient. Once the size of the interbody spacer 10, 300 is known, the surgeon then selects the appropriately sized working tube 9 that is sized and dimensioned to accommodate the selected interbody spacer 10, 300. The surgeon may perform an endoscopic foraminoplasty to enlarge the corridor and provide expanded access to the intervertebral disc and intervertebral disc space.

The surgeon then inserts the working tube 9 and fixes the tube in place, as is known in the field of the disclosure. The surgeon performs a discectomy and other procedures, such as end plate preparation, in order to prepare the intervertebral space for receiving the interbody spacer 10, 300.

Once the intervertebral space has been adequately prepared, the surgeon adjusts the actuator 200, 500 so that the interbody device 10, 300 is in the collapsed configuration. For embodiments including a screw plate 600, the screw plate is separated from cage 312. The surgeon delivers cage 12, 312 of device 10, 300 through working tube 9 and into the intervertebral space. According to one embodiment, cage 12, 312 is inserted between the adjacent vertebral bodies at about a variable angle based on anatomy within Kambin's triangle (20-50 deg angle) in relation to the coronal plane. The surgeon confirms correct placement of cage 12, 312, for example, by using a fluoroscope, CT scanner, or the like to visualize radio-opaque markers embedded in one or more of the spacers. Once the initial placement of the device is correct, the surgeon inserts a rotary driving device through working tube 9 to engage with drive 212a, 512a of actuator 200, 500. The surgeon uses a driving device to expand cage 12, 512. According to one embodiment, the surgeon monitors the positions of the spacers using a radiographic imaging device to confirm that all four spacers are in contact with the cortical bony rim of the adjacent end plates.

In the embodiment with a screw plate, such as was discussed with respect to FIGS. 16-19, the surgeon delivers screw plate 600 through working tube 9 and engages the screw plate with posterior spacer 350 by inserting pins 603 into openings 359. Locking protrusion 605 of the screw plate 600 engages the mating feature 512a of the head 512 such that the actuator 500 is locked in place.

Bone screws 316 are then delivered through working tube 9 and inserted through bone screw openings 612 in screw plate 600. The surgeon drives bone screws 316 into the adjacent vertebral bodies so that the interbody device 300 is secured and will not postoperatively migrate. According to one embodiment, the surgeon delivers bone graft material into opening 18.

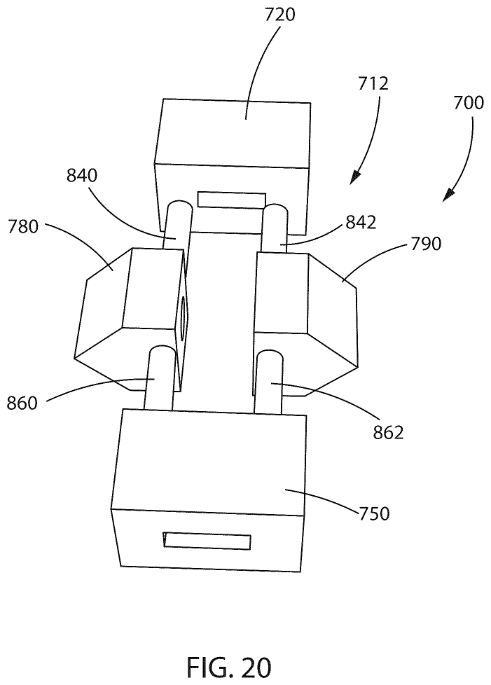

FIG. 20 show interbody device 700 according to another embodiment of this disclosure. Interbody device 700 includes cage 712 having an anterior spacer 720, a posterior spacer 750, lateral spacers 780, 790, and hinge members 840, 860, 842, 862. In an exemplary embodiment, radiographic markers are embedded within one or more components of cage 712. Cage 712 is arranged and assembled substantially similar to cage 12 described above and for ease of description is not repeated.

The anterior spacer 720 is substantially similar to the anterior spacer 20 described above, except that anterior spacer 720 does not include bore 25.

The posterior spacer 750 is substantially similar to the posterior spacer 50 described above, except that posterior spacer 750 does not include aperture 50.

Lateral spacers 780, 790 are substantially similar to lateral spacers 80, 90 described above with reference to FIGS. 9-10 and a detailed description thereof is not repeated.

Hinge members 840, 842, 860, 862 are substantially similar to hinge members 140, 142, 160, 162 described above with reference to FIGS. 11-14 and a detailed description thereof is not repeated.

The surgical procedure and placement of interbody device 700 is substantially similar to the placement of interbody devices 10, 300 described above, except that device 700 does not include an actuator for expanding interbody device 700. Instead, the surgeon manipulates the spacers individually within the intervertebral space until cage 712 is expanded and the spacers are in the desired location. The spacers may be manipulated by the surgeon using a variety of tools.

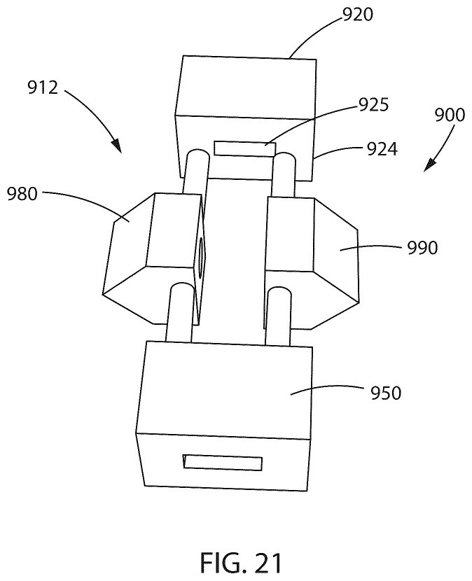

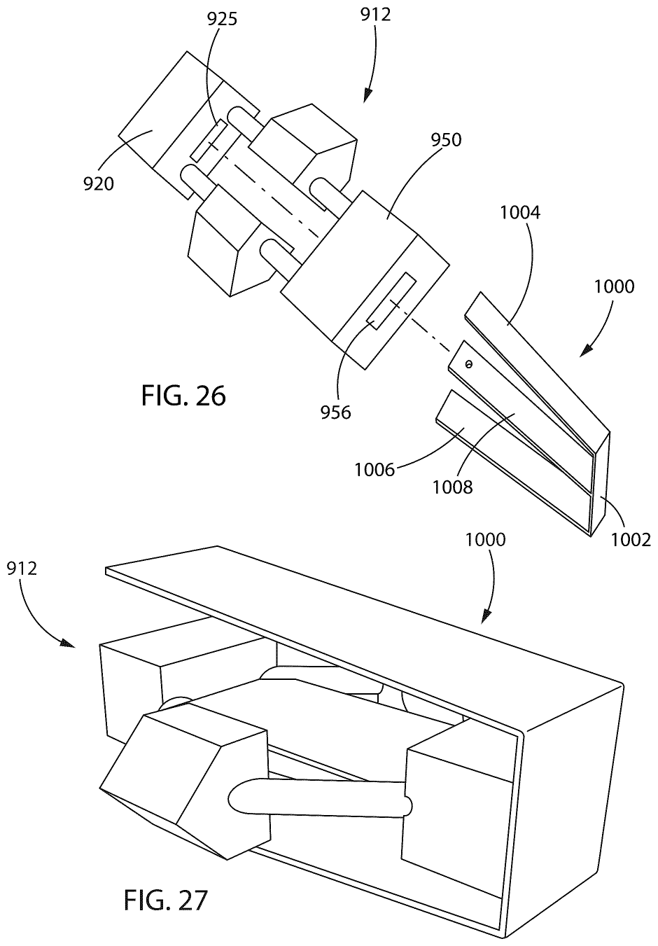

According to a further embodiment of the disclosure, a bone staple may be provided to secure the interbody device to one or more adjacent vertebrae. Referring now to FIGS. 21-27, an exemplary embodiment of an interbody device 900 according to the present disclosure is shown. Interbody device 900 is substantially similar to interbody device 700 described above except that anterior spacer 920 and posterior spacer 950 include coupling features for coupling the bone staple to cage 912 which will be described below in more detail.

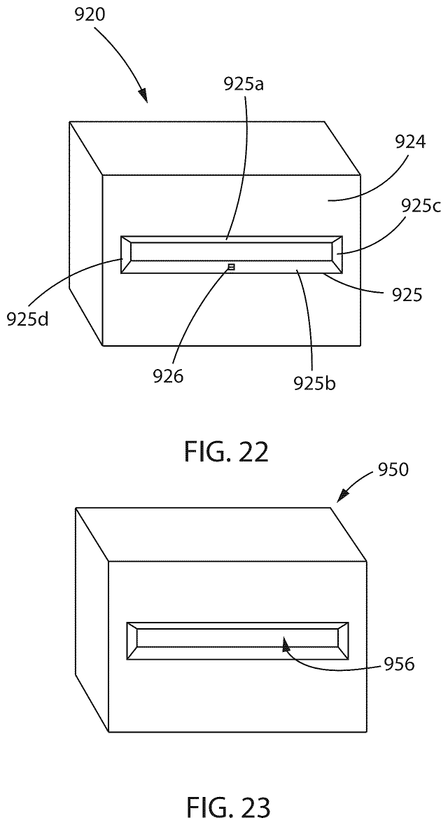

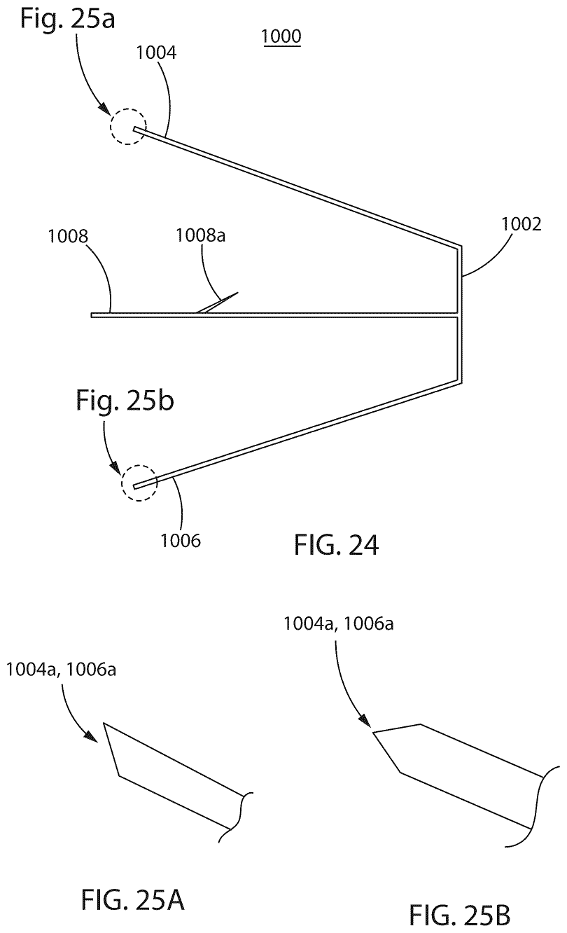

In the exemplary embodiment of FIG. 22, the rear wall 924 of the anterior spacer 920 includes a receiving channel 925 that is generally centrally located. Receiving channel 925 is configured and dimensioned to receive a bone staple 1000 that will be discussed in greater detail below in regard to FIG. 24 Receiving channel 925 is defined by a top wall 925a, bottom wall 925b and two side walls 925c, 925d. In one embodiment, any combination of top wall 925a, bottom wall 925b and/or side walls 925c, 925d include a retention feature 926. In yet another embodiment, more than one retention feature is present on the top wall 925a, bottom wall 925b, and/or side walls 925c, 925d. As shown in FIG. 22, in this embodiment the bottom wall 925b includes retention feature 926. The retention feature 926 is sized and dimensioned such that a flexible projection 1008a of bone staple 1000 will mate with the retention feature 926 thereby securing the bone staple 1000 to cage 912 as will be described in more detail below.

Referring now to FIG. 23, the posterior spacer 950 includes a receiving slot 956 that is generally centrally located and extending through the spacer from the posterior to the anterior surface thereof. Receiving slot 956 is sized and dimensioned to receive bone staple 1000.

According to one embodiment, bone staple 1000 includes two piercing members 1004, 1006 and a securing member 1008. In the exemplary embodiment shown in FIGS. 24-25b, the two piercing members 1004, 1006 are substantially planar plate-like structures. In one embodiment, the two piercing members 1004, 1006 are monolithically formed into the transverse bridge 1002. In another embodiment, the two piercing members 1004, 1006 are secured to the transverse bridge 1002 using, for example, welds, mechanical fasteners, or adhesives. The two piercing members extend away from the transverse bridge 1002 at an angle ranging from 90 degrees to about 135 degrees. In one embodiment, the sides of the piercing members 1004, 1006 are smooth. In another embodiment, one or more sides of the piercing members 1004, 1006 are serrated or toothed. In an embodiment, the piercing end of the piercing members 1004, 1006 have bone penetrating features 1004a, 1006a, such as, for example, a wedge-like shape for easier insertion of the staple into a vertebrae. In one non-limiting example, as shown in FIG. 25A, the penetrating features 1004a, 1006a may consist of a single angled surface. In another non-limiting embodiment, as shown in FIG. 25B, the penetrating features 1004a, 1006a may consist of more than one angled surface.

Continuing to refer to FIG. 24, in the exemplary embodiment shown, securing member 1008 is a substantially planar plate-like structure disposed between piercing members 1004, 1006. In one embodiment, securing member 1008 is monolithically formed into the transverse bridge 1002. In another embodiment, securing member 1008 is secured to the transverse bridge 1002 using, for example, welds, mechanical fasteners, or adhesives. The securing member 1008 is generally centrally located and extends away from the transverse bridge 1002. In an exemplary embodiment, securing member 1008 includes a flexible projection 1008a that is sized and dimensioned to fit into the retention feature 926 of anterior spacer 920. It is envisioned that flexible projection 1008a may be a cantilever and/or snap-fitting extension. It is contemplated within this disclosure, flexible projection 1008a may be located on along any side of the securing member 1008. It is also contemplated within this disclosure that there may be more than one flexible projection 1008a.

The procedure for placing cage 912 in the patient is substantially similar as the placement of cage 712, except for once the surgeon positions cage 912, the surgeon uses bone staple 1000 to secure cage 912 such that the cage remains in place without any movement and/or shifting. Prior to inserting the bone staple 1000, the surgeon selects the appropriately sized staple. It is preferred that the transverse bridge 1002 is flush with the posterior spacer 950 as the securing member engages the retention feature 926.

As shown in FIG. 26, securing member 1008 is inserted through receiving slot 956 of the posterior spacer 950. As the securing member 1008 is inserted therethrough, the piercing members 1004, 1006 start to engage the adjacent vertebrae. In an exemplary embodiment, the securing member 1008 extends beyond piercing members 1004, 1006 such that securing member 1008 is at least partially inserted through receiving slot 956 prior to piercing members 1004, 1006 engagement of the vertebrae.

As the piercing members 1004, 1006 start to engage the vertebrae, the surgeon may require the assistance of a variety of tools well known in the art, such as, for example, a mallet, to assist in driving bone staple 1000 with sufficient force that piercing members 1004, 1006 are driven into the vertebrae located anteriorly and posteriorly of the disc space. As the bone staple 1000 continues to be driven through cage 912 and into the adjacent vertebrae, securing member 1008 advances through the receiving slot 956 and into the receiving channel 925 until the flexible projection 1008a of the securing member 1008 engages the retention feature 926.

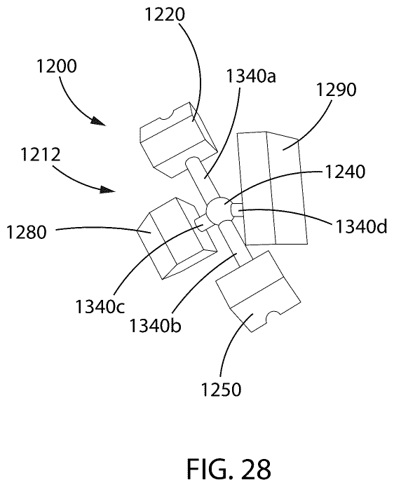

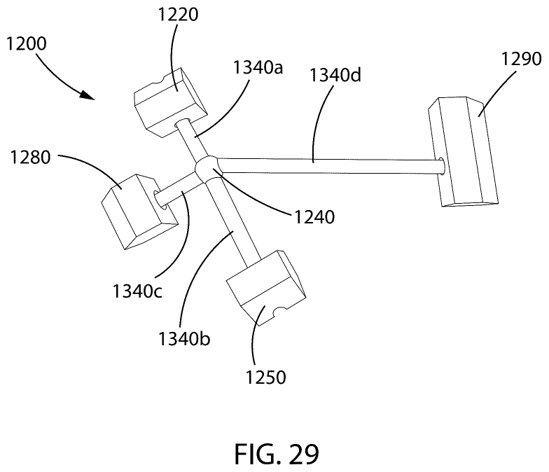

According to a further embodiment of the disclosure, an exemplary embodiment of interbody device 1200 is provided as shown in FIGS. 28-35c. Interbody device 1200 includes cage 1212 having an anterior spacer 1220, posterior spacer 1250, lateral spacers 1280, 1290, hinge members 1340a, 1340b, 1340c, 1340d, and a central hub 1400. In an exemplary embodiment, radiographic markers are embedded within one or more components of cage 1212. As shown in FIGS. 28-29, anterior spacer 1220 is connected to central hub 1400 by hinge member 1340a. Posterior spacer 1250 is connected to central hub 1400 by hinge member 1340b. Lateral spacer 1280 is connected to central hub by hinge member 1340c. Lateral spacer 1290 is connected to central hub by hinge member 1340d. In one embodiment, the hinge members are expandable, as shown in FIG. 29, such that the surgeon is able to move the spacers to the desired location within the intervertebral space. Exemplary embodiments in FIGS. 28 and 29 show four spacers 1220, 1250, 1280, and 1290. A greater of fewer number of spacers, each connected with central hub 1240 are within the scope of the disclosure.

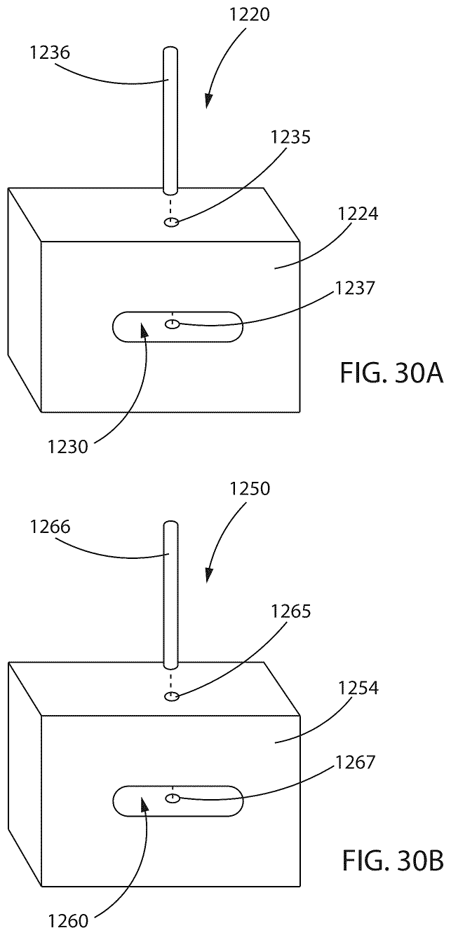

Referring to FIG. 30A, an exemplary embodiment of anterior spacer 1220 is shown. Anterior spacer 1220 is substantially similar to anterior spacer 20 discussed above except anterior spacer 1220 does not include a threaded bore and only has a singular hinge member receiving channel 1230. Hinge member receiving channel 1230 is generally centrally located on the rear wall 1224 and has a horizontally oblong shape. It is contemplated within this disclosure, the receiving channel 1230 may be of any suitable shape that allows anterior spacer 1220 to move along a horizontal plane in relation to hinge member 1340a as will be discussed in more detail below. Similar to anterior spacer 20, anterior spacer 1220 includes an aperture 1235 therethrough the top wall and a bore 1237 within the hinge member receiving channel 1230 coaxially aligned with aperture 1235. Aperture 1235 is dimensioned and configured to receive pin 1236. Bore 1237 is dimensioned and configured for receiving at least a portion of pin 1236 such that hinge member 1340a is rotatably connected to anterior spacer 1220.

Referring to FIG. 30B, an exemplary embodiment of posterior spacer 1250 is shown. Posterior spacer 1250 is substantially similar to posterior spacer 50 discussed above except posterior spacer 1250 has only a singular hinge member receiving channel 1260. Hinge member receiving channel 1260 is generally centrally located on the front wall 1254 and has a horizontally oblong shape. It is contemplated within this disclosure, the receiving channel 1260 may be of any suitable shape that allows for posterior spacer 1250 to move along a horizontal plane in relation to hinge member 1340b as will be discussed in more detail below. Similar to posterior spacer 50, posterior spacer 1250 includes an aperture 1265 therethrough the top wall and a bore 1267 within the hinge member receiving channel 1260 coaxially aligned with aperture 1265. Aperture 1265 is dimensioned and configured to receive pin 1266. Bore 1267 is dimensioned and configured for receiving at least a portion of pin 1266 such that hinge member 1340b is rotatably connected to anterior spacer 1250.

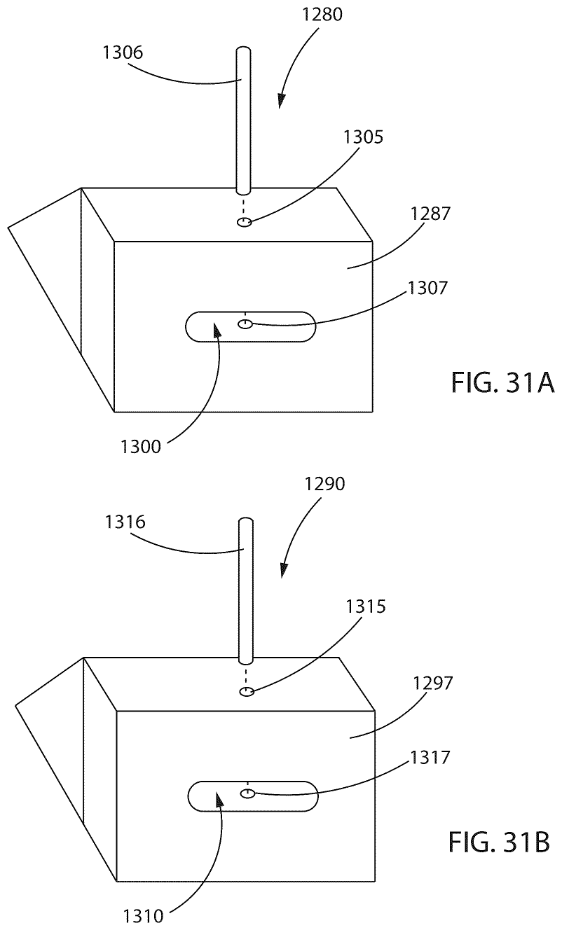

Referring to FIG. 31A, an exemplary embodiment of lateral spacer 1280 is shown. Lateral spacer 1280 is substantially similar to lateral spacer 80 discussed above except lateral spacer 1280 has only a singular hinge member receiving channel 1300. Hinge member receiving channel 1300 is generally centrally located on the side wall 1287 and has a horizontally oblong shape. It is contemplated within this disclosure, the receiving channel 1300 may be of any suitable shape that allows for lateral spacer 1280 to move along a horizontal plane in relation to hinge member 1340c as will be discussed in more detail below. Similar to lateral spacer 80, lateral spacer 1280 includes an aperture 1305 therethrough the top wall and a bore 1307 within the hinge member receiving channel 1300 coaxially aligned with aperture 1305. Aperture 1305 is dimensioned and configured to receive pin 1306. Bore 1307 is dimensioned and configured for receiving at least a portion of pin 1306 such that hinge member 1340c is rotatably connected to lateral spacer 1280.

Referring to FIG. 31bB, an exemplary embodiment of lateral spacer 1290 is shown. Lateral spacer 1290 is substantially similar to lateral spacer 1280 and for ease of description will not be further described.

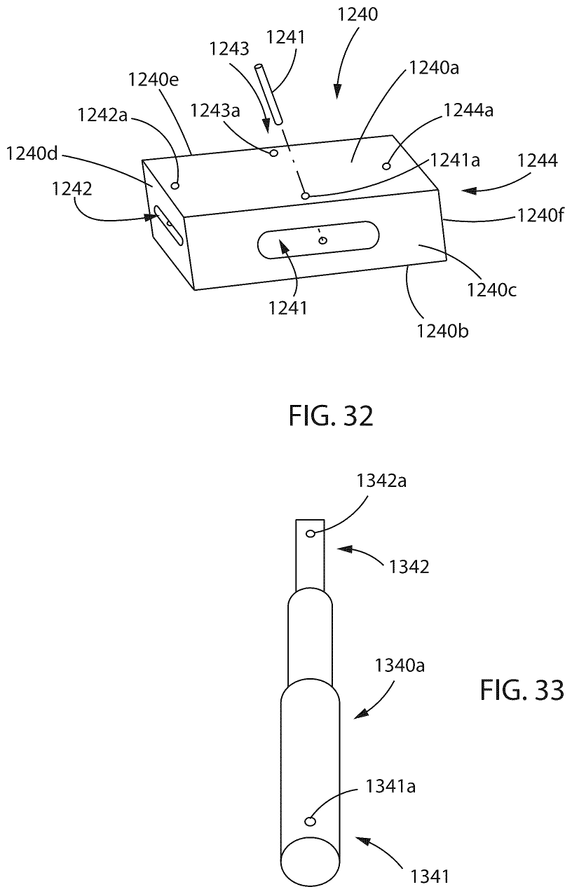

Referring to FIG. 32, an exemplary embodiment of central hub 1240 is shown. Central hub 1240 has a top wall 1240a, bottom wall 1240b, and side walls 1240c, 1240d, 1240e, 1240f. The side walls include hinge member receiving channels 1241, 1242, 1243, and 1244. In the embodiment shown, hinge member receiving channels 1241, 1242, 1243, and 1244 are horizontally oblong shape. However, it is contemplated within this disclosure that hinge member receiving channels 1241, 1242, 1243, and 1244 may be of any suitable shape that allows for hinge members to move along on a horizontal plane. Hinge member receiving channels 1241, 1242, 1243, 1244 are sized to receive one end of hinge members 1340a, 1340b, 1340c, 1340d. The top wall 1240a of the central hub 1240 has apertures 1241a, 1242a, 1243a, 1244a therethrough that are dimensioned and configured to receive retaining pins 1241 (only pin 1241 in aperture 1241a is shown). Within each hinge member receiving channel is a respective bores that are coaxially aligned with the respective aperture 1241a, 1242a, 1243a, 1244a and is configured and dimensioned to receive at least a portion of the respective retaining pins 1241.

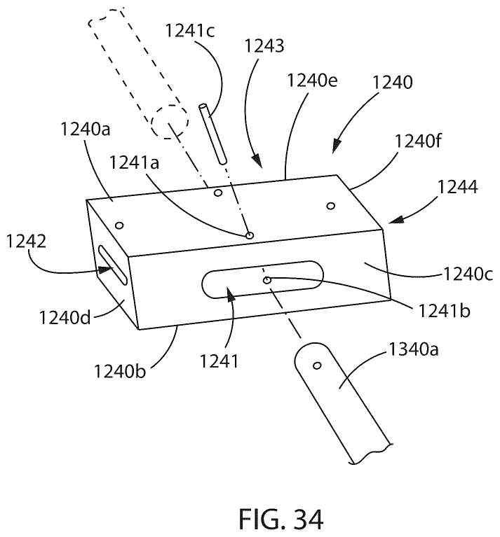

Referring now to FIGS. 33 and 34, an exemplary embodiment of hinge member 1340a is shown. In one embodiment, hinge member 1340a has a circular cross section. Hinge member 1340a has a first end 1341 and a second end 1342. First end 1341 of the hinge member 1340a, is dimensioned and configured to be received by the hinge member receiving channel 1241 of central hub 1240. First end 1341 of hinge member 1340a has an aperture 1341a that is configured and dimensioned to receive pin 1241 such that hinge member 1340a is rotatably connected to central hub 1240. Second end 1342 of hinge member 1340a is dimensioned and configured to be received by hinge member receiving channel 1230 of the anterior spacer 1220. Likewise, second end 1342 of hinge member 1340a has an aperture 1342a that is configured and dimensioned to receive a pin 1241 such that hinge member 1340a is rotatably connected to the hinge member receiving channel 1230 of the anterior spacer 1220. According to an embodiment, as shown in FIG. 33, hinge member 1340a is extendable, for example, telescopically, such that the distance between the respective spacer and central hub 1240 may be increased and/or decreased as desired by the surgeon and/or dictated by the anatomy of the patient.

Hinge members 1340b, 1340c, and 1340d are substantially similar to hinge member 1340a described above and for case of description are not described further.

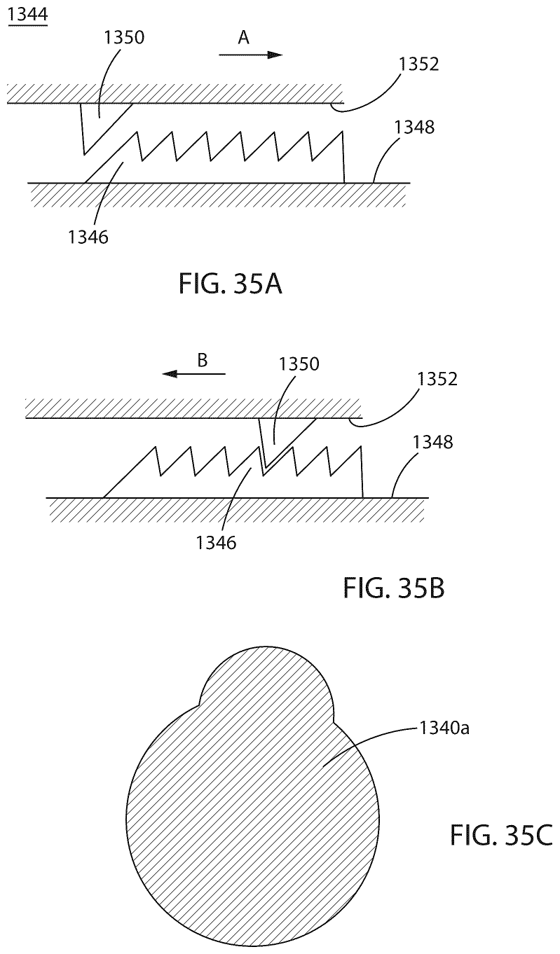

To keep the extendable hinge members in their final extended state after deployment, a one-way latch may be used to lock the adjacent segments. FIG. 35A shows latch 1344 according to one embodiment, in a first position, for locking the extendable hinge members at a selected length. The latch 1344 may consist of one or more grooves 1346 associated with a first segment 1348 and a tooth 1350 associated with a second, adjacent segment 1352. As the extendable hinge members are expanded, the second segment 1352 moves in a first direction A relative to the first segment 1348. The tooth 1350 and the grooves 1346 are aligned so as to engage when the extendable hinge members are extended. Once the tooth 1350 engages groove 1346, as shown in FIG. 35B, the second segment 1352 may not move in a second direction B relative to the first segment 1348. Accordingly, the extendable hinge members are free to extend but may not collapse once extended. Of course, other one-way latches may be used to lock the segments that may make up the extendable hinge members. FIG. 35C illustrates one possible cross-section of a telescoping segment of an extendable hinge member. This “rail” design permits room for sliding and positioning of a one-way latch, like the one shown in FIGS. 33A and 33B, between telescoping segments of a hinge member.

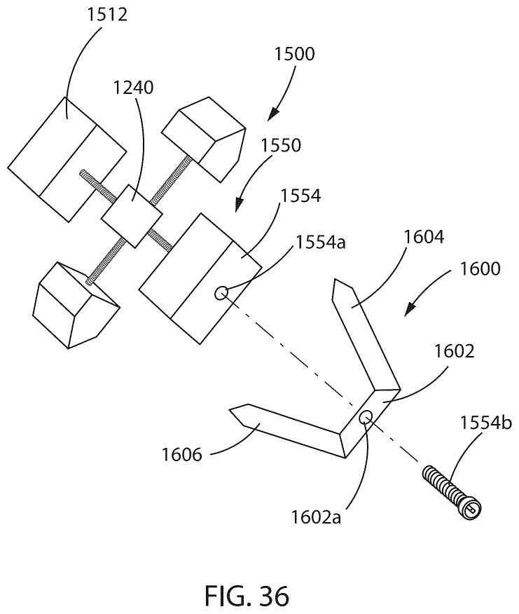

According to a further embodiment of the disclosure, a bone staple may be provided to secure interbody device to one or more adjacent vertebrae. Referring now to FIG. 36, an exemplary embodiment of an interbody device 1500 according to the present disclosure is shown. Interbody device 1500 is substantially similar to interbody device 1200 described above except that posterior spacer 1550 includes a coupling feature for coupling the bone staple to cage 1512 as will be described in more detail below.

In the exemplary embodiment of FIG. 36, the rear wall 1554 of posterior spacer 1550 includes a coupling feature 1554a. In the exemplary embodiment shown, the coupling feature 1554a is a threaded bore that is generally centrally located on rear wall 1554. Threaded bore 1554a is sized and dimensioned to accommodate a mechanical fastener 1554b, for example, a bolt, to secure a bone staple 1600 to cage 1512, as will be discussed in further detail below.

According to one embodiment, bone staple 1600 includes a transverse bridge 1602 and two piercing members 1604, 1606. Transverse bridge 1602 may have a circular cross section. However, it is contemplated in this disclosure that transverse bridge 1602 may have any cross-sectional configuration. Transverse bridge 1602 may include an aperture 1602a therethrough used for securing bone staple 1600 to cage 1512 as will be discussed in further detail below. In one embodiment, the two piercing members 1604, 1606 are monolithically formed into the transverse bridge 1602. In another embodiment, the two piercing members 1604, 1606 are secured to the transverse bridge 1602 using, for example, welds, mechanical fasteners, or adhesives. The two piercing members 1604, 1606 have a circular cross section. However, one skilled in the art will appreciate there can be several variations of bone staples. For example, the piercing members can be in various cross-sectional forms such as circular, rhombus, square, triangular, and rectangular.

The procedure for placing cage 1512 in the patient is substantially similar as the placement of cage 1212. Once the surgeon positions cage 1512 so that spacers 1520, 1550, 1580 and 1590 are positioned adjacent to selected regions of the adjancent vertebrae, the surgeon uses bone staple 1600 to secure cage 1512 such that cage 1512 remains in place without any movement and/or shifting.

The various embodiments within this disclosure have been described with four spacers. However, it is envisioned that more or fewer spacers may be used. Moreover, it is also envisioned that any of the spacers may be of a wedge shape or rectangular shape as disclosed above.

As shown throughout the drawings, like reference numerals designate like or corresponding parts. While illustrative embodiments of the present disclosure have been described and illustrated above, it should be understood that these are exemplary of the disclosure and are not to be considered as limiting. Additions, deletions, substitutions, and other modifications can be made without departing from the spirit or scope of the present disclosure. Accordingly, the present disclosure is not to be considered as limited by the foregoing description.

What is claimed is:

1. A device for facilitating a spinal fusion procedure comprising:
an anterior spacer;
a posterior spacer;
a first lateral spacer;
a second lateral spacer; and
first, second, third, and fourth hinge members, wherein the first hinge member extends between and is rotatably coupled with the anterior spacer and the first lateral spacer, wherein the second hinge member extends between and is rotatably coupled with the first lateral spacer and the posterior spacer, wherein the third hinge member extends between and is rotatably coupled with the posterior spacer and the second lateral spacer, wherein the fourth hinge member extends between and is rotatably coupled with the anterior spacer and second lateral spacer.

2. The device of claim 1, further comprising an actuator extending between the anterior spacer and the posterior spacer, wherein actuation of the actuator in a first direction moves the anterior and posterior spacers away from one another to place the device in a collapsed configuration and actuation of the actuator in a second direction moves the anterior and posterior spacers toward one another to place the device in an expanded or partially expanded configuration.

3. The device of claim 2, wherein the actuator is a rod extending between the anterior and posterior spacers and adapted to rotate with respect to the device, wherein the anterior or posterior space comprises a threaded opening, wherein at least a portion of the rod comprises screw threads along a distal portion of the rod, wherein the screw thread engage the threaded opening, and wherein rotation of the rod in the first direction draws the anterior and posterior spacers toward one another.

4. The device of claim 3, wherein the anterior or posterior spacer further comprises a threaded sleeve forming a portion of the threaded opening.

5. The device of claim 2, wherein, in the collapsed configuration a lateral distance defined by the lateral spacers is adapted for delivery through an endoscope or working tube.

6. The device of claim 2, wherein, in the expanded or partially expanded configuration, one or more of the anterior spacer, posterior spacer, and lateral spacers are adapted to be placed between cortical bony rims of the endplates of adjacent vertebrae in an MIS procedure.

7. The device of claim 3, wherein the rod includes a tool engagement at an end of the rod opposite the screw threads, wherein the tool engagement is adapted to be coupled with a tool for rotation of the rod to actuate the actuator.

8. The device of claim 7, further comprising a screw plate, wherein the screw plate includes one or more bone screw apertures, wherein the screw plate is adapted to be connected with the anterior or posterior spacer.

9. The device of claim 8, wherein the screw plate further comprises a locking protrusion, wherein, when the screw plate is engaged with the anterior or posterior spacer, the locking protrusion engages with the tool engagement to prevent rotation of the rod.

10. The device of claim 8, wherein the screw plate comprises one or more bone screw apertures, wherein the apertures are adapted to hold a respective bone screw, and wherein the bone screws are adapted to connect the device with adjacent vertebrae using MIS techniques.

11. The device of claim 1, wherein one or more surfaces of the anterior, posterior, and lateral spacers comprise a surface coating or surface texture, and wherein the coating or texture is adapted to facilitate ongrowth of bone tissue.

12. The device of claim 1, wherein the anterior spacer includes a receiving channel having a retention feature.

13. The device of claim 12, further comprising a bone staple having at least two piercing members and a securing member disposed between the piercing members, and wherein the securing member is adapted to engage with the receiving channel, and wherein the securing member includes at least one flexible projection configured to engage with the at least one retention feature.

14. A device for facilitating a spinal fusion procedure comprising:
a plurality of spacers;
a central hub; and
a plurality of hinge members, wherein respective ones of the hinge members rotatably couple respective ones of the spacers with the central hub, and wherein the spacers are adapted to be positioned with respect to adjacent vertebrae during the procedure by rotating the respective hinge member relative to the hub.

15. The device of claim 14, wherein the plurality of spacers comprise:
an anterior spacer;
a posterior spacer;
a first lateral spacer; and
a second lateral spacer.

16. The device of claim 14, wherein one or more of the plurality of hinge members is extendable to a selected length.

17. The device of claim 16, wherein the extendable hinge member comprises a locking mechanism, wherein the length of the hinge member is adapted to be locked to the respective spacer between cortical bony rims of endplates of the adjacent vertebrae.
